(19) United States
(12) Patent Application Publication
(10) Pub. No.: US 2024/0257339 A1
(43) Pub. Date: Aug. 1, 2024

(54) METHOD FOR GENERATING RARE MEDICAL IMAGES FOR TRAINING DEEP-LEARNING ALGORITHMS

(71) Applicant: Quantum Surgical, Montpellier (FR)

(72) Inventors: Michael GIRARDOT, Castelnau-le-Lez (FR); Lucien BLONDEL, Montpellier (FR); Bertin NAHUM, Castelnau-le-Lez (FR); Fernand BADANO, Lyon (FR)

(21) Appl. No.: 18/290,385

(22) PCT Filed: May 6, 2022
(86) PCT No.: PCT/FR2022/050869

§ 371 Date: Nov. 13, 2023

(30) Foreign Application Priority Data
May 11, 2021 (FR) FR2104970

(51) Int. Cl. G06T 7/00; G06T 3/40; G06T 5/50; G06T 7/11; G06V 10/25; G16H 30/40

(52) U.S. Cl. CPC G06T 7/0012; G06T 3/40; G06T 5/50; G06T 7/11; G06V 10/25; G16H 30/40; G06T 2207/20084; G06T 2207/20221; G06T 2207/30096

(57) ABSTRACT

The invention relates to a method for generating synthetic medical images representing an anatomy of interest and an anomaly within said anatomy of interest. The method comprises generating majority segmentation masks associated with real medical images without anomaly, generating minority segmentation masks associated with real medical images with an anomaly, training a neural network to generate a synthetic medical image on the basis of a segmentation mask, generating artificial segmentation masks on the basis of the majority and minority segmentation masks by combining a segmentation of the anatomy of interest by a majority segmentation mask with a segmentation of the anomaly by a minority segmentation mask, and generating synthetic medical images on the basis of the artificial segmentation masks and using the previously trained neural network.

### FIELD OF THE INVENTION

The present application relates to the field of generating synthetic medical images exhibiting rare anatomical anomalies using an artificial neural network. The generated synthetic images are intended to be used to train a machine learning algorithm aiming to detect or to characterize an anomaly within an anatomy of interest visible on a medical image, for example, a deep neural network for classifying or for segmenting an anomaly.

### PRIOR ART

Training a machine learning algorithm, and more specifically training an artificial neural network, requires a large amount of data in the training set in order to achieve good prediction quality. Underrepresenting a rare class of data in the training set actually affects the prediction bias observed for one class over another.

This problem is particularly important in the medical field, for example, for training a deep neural network aiming to detect or to characterize an anomaly within an anatomy of interest visible on a medical image. Indeed, it is difficult to gather large amounts of medical images due to the rarity of the illnesses, the confidentiality of the patients, the efforts and expenses required to undertake medical imaging operations, etc.

Several existing solutions aim to artificially increase the amount of data corresponding to rare classes in the training set by creating synthetic medical images exhibiting rare anatomical anomalies. However, in the solutions of the prior art, the synthetic images that are obtained are generally not different enough from the real images from which they are generated, and they do not allow optimal training of a deep neural network for classification or for segmentation. Indeed, there is then a risk of overlearning a particular class at the expense of other classes. Overlearning denotes the phenomenon of the loss of generalization of the predictions of an algorithm: the predictions are good with respect to the data of the training set, but they are poor with respect to new data.

Patent application US 2019/0370969 A1 teaches, for example, training an algorithm for classifying a tumour with synthetic images generated by “Generative Adversarial Networks” (or GAN). The classification algorithm is trained using synthetic images mixed with real images. Several generative adversarial neural networks are required to produce images of different classes of tumours. The method for training the classification algorithm comprises a method for adjusting the weight of the images as a function of the learning performance of the algorithm, which allows the learning of difficult cases to be enhanced, in particular the cases that are underrepresented in the training set. The major disadvantage of this method is the requirement to train a plurality of generative adversarial neural networks in order to produce different classes of synthetic images, which is technically difficult. Another limitation of this approach is that the diversity of the synthetic images that are obtained is limited by the diversity of the images used to train the generative adversarial neural networks. The synthetic images of rare classes are difficult to produce if they are not initially present in the sets of training data of the generative adversarial neural networks.

Some solutions implement at least two generative adversarial neural networks that are interdependent of each other. Such an approach significantly increases the technical complexity, particularly since the optimization of one of the generators depends on the optimization of the other.

Therefore, a requirement still exists for a solution that is relatively simple to implement in order to create a large number and a wide variety of images of rare anatomical anomalies for training a learning algorithm aiming to detect or to characterize an anomaly.

### DISCLOSURE OF THE INVENTION

The aim of the methods and devices disclosed in the present application is to overcome all or some of the disadvantages of the prior art, particularly those described above.

To this end, and according to a first aspect, a method for generating synthetic medical images representing an anatomy of interest and an anomaly within said anatomy of interest is proposed. The method comprises:
generating majority segmentation masks, each majority segmentation mask being associated with a majority real medical image representing the anatomy of interest of a patient without an anomaly;
generating minority segmentation masks, with each minority segmentation mask being associated with a minority real medical image representing the anatomy of interest of a patient with an anomaly;
training a neural network in order to generate a synthetic medical image from a segmentation mask;
generating artificial segmentation masks from majority segmentation masks and from minority segmentation masks, the generation of an artificial segmentation mask comprising combining a segmentation of the anatomy of interest of a majority segmentation mask with a segmentation of the anomaly of a minority segmentation mask;
generating synthetic medical images from the artificial segmentation masks using the previously trained neural network.

The anatomy of interest can correspond to an organ (for example, the liver, the pancreas, the gall bladder, a lung or a kidney) or to another anatomical structure (for example, a bone or a blood vessel). An anomaly present within the anatomy of interest generally corresponds to a lesion, such as, for example, a tumour, a cyst, an ablation zone, an aneurysm, etc. An ablation zone corresponds to a lesion that has undergone ablation treatment using a known method (microwave, laser, radiofrequency, etc.). It involves a necrotic area.

A “real medical image” is understood to be a medical image acquired on a patient using a medical imaging device, for example, by tomodensitometry (CT (Computerized Tomography) scan), by Positron Emission Tomography (PET scan), by Magnetic Resonance Imaging (MRI), by ultrasound or by x-rays.

The terms “majority” and “minority” are used since generally there is a significantly greater number of medical images representing an anatomy of interest without an anomaly than medical images representing an anatomy of interest with an anomaly.

A “synthetic” medical image is, by contrast, artificially generated by the neural network. A neural network is a hardware and/or software computer system, the operation of which is inspired by the neurons of the human brain. It is a variety of “deep learning” technology, which itself forms part of the machine learning algorithms. The machine learning algorithms form a category of the field of artificial intelligence.

The neural network is trained to generate a synthetic image from a segmentation mask associated with a real medical image. The majority segmentation masks and the minority segmentation masks can be used to train the neural network. A segmentation mask is an image, each voxel of which provides particular information relating to an element shown at the position of the voxel on the real medical image. A voxel thus can assume a particular numerical value associated with the element shown at the position of the voxel on the real medical image (a specific numerical value is, for example, defined for a healthy part of the anatomy of interest, and a different numerical value is defined for an anomaly). It should be noted that the term “voxel” is generically used to define a particular zone of an image (a voxel identifies a position of said zone on the image and assumes a value representing what is shown in said zone on the image). It can involve a two-dimensional or three-dimensional image. If it involves a two-dimensional image, the term “voxel” then assumes the same meaning as the term “pixel”.

Various types of neural networks can be contemplated for generating a synthetic image from a segmentation mask. For example, it is possible to contemplate using an autoencoder (“Variational Autoencoder” or VAE) type neural network. However, using a generator of a pair of generative adversarial neural networks (GAN) is preferable. These types of neural networks actually allow images to be generated that are highly realistic. A GAN is a generative model where two neural networks are set to compete in a zero-sum game scenario. The first network, the generator, generates an image, and its adversary, the discriminator, attempts to detect whether the generated image is real or if it is a synthetic image generated by the generator.

Generating a synthetic image from a segmentation mask, and not from random “noise”, as is generally the case in the prior art, is advantageous. The use of a segmentation mask as input for the neural network allows greater control to be provided with respect to the synthetic image produced by the neural network.

The invention is based on generating artificial segmentation masks from a set of majority segmentation masks associated with majority real medical images and from a set of minority segmentation masks associated with minority real medical images. In order to generate an artificial segmentation mask, the segmentation of the anatomy of interest of a majority segmentation mask is combined with the segmentation of the anomaly of a minority segmentation mask. The segmentation of the anomaly is integrated, for example, in the majority segmentation mask at various positions in the anatomy of interest, and in various orientations. It is also possible to contemplate deforming the segmentation of the anomaly of interest before integrating it into the majority segmentation mask. Each majority segmentation mask can be combined with each minority segmentation mask.

Such arrangements allow a very wide diversity of artificial segmentation masks to be generated. This then subsequently allows a very wide diversity of synthetic medical images to be generated exhibiting anatomical anomalies.

This diversity of the synthetic medical images allows a set of training images to be generated that is particularly effective for a machine learning algorithm aiming to detect or to characterize an anomaly in the anatomy of interest of a patient on a real medical image. The set of training images can comprise both real medical images and synthetic medical images. The number of medical images exhibiting an anomaly and the variety of the anomalies are considerably increased by virtue of the wide variety of artificial segmentation masks. Preferably, the set of training images comprises a number of images with an anomaly that is substantially equal to the number of images without an anomaly. This thus allows the phenomenon of overlearning on a particular class of anomaly to be avoided.

Furthermore, the technical complexity of the proposed solution is relatively limited. In particular, generating artificial segmentation masks does not require the use of a learning algorithm. The method proposed for generating synthetic medical images therefore requires at most a single GAN (assuming that the neural network used to generate the synthetic images is a GAN).

It should further be noted that the proposed method is not limited to the generation of synthetic medical images representing a single anatomy of interest with a single anomaly within said anatomy of interest. In other words, the method can also allow synthetic medical images representing an anatomy of interest with a plurality of anomalies within the anatomy of interest (the anomalies potentially being of different natures) to be generated. Thus, the method can allow synthetic medical images representing a plurality of different anatomies of interest potentially with a plurality of anomalies within each represented anatomy of interest to be generated. With this aim, the artificial segmentation masks can be generated from combinations of majority segmentation masks corresponding to different anatomies of interest and of minority segmentation masks corresponding to different types of anomalies.

In particular embodiments, the method can further comprise one or more of the following feature(s), taken individually or according to all the technically possible combinations.

In particular embodiments, the generation of an artificial segmentation mask further comprises transforming the segmentation of the anomaly of the minority segmentation mask.

In particular embodiments, the transformation of the segmentation of the anomaly corresponds to a rotation, a magnification, a reduction, a deformation and/or a movement of the segmentation of the anomaly.

In particular embodiments, the generation of an artificial segmentation mask further comprises checking that the segmentation of the anomaly relative to the segmentation of the anatomy of interest meets a particular criterion.

This involves, for example, checking that certain particular constraints are met in order to filter the unrealistic combinations. These constraints relate to, for example, the location of the anomaly within the organ of interest or relative to other organs or other anatomical structures.

In particular embodiments, a segmentation mask comprises a set of voxels, with each voxel corresponding to a zone of the real medical image with which the segmentation mask is associated, with each voxel being associated with a numerical value encoding what is shown by said zone on the real medical image, and the step of generating an artificial segmentation mask comprises:
selecting a majority segmentation mask and a minority segmentation mask;
identifying, on the selected minority segmentation mask, a set of voxels, the numerical value of which encodes the anomaly;
replacing, on the selected majority segmentation mask, the numerical value of the voxels identified by the numerical value encoding the anomaly.

In particular embodiments, the neural network used to generate a synthetic medical image is a generator neural network, and the training of the generator neural network is implemented using a discriminator neural network, with the generator neural network and the discriminator neural network forming a pair of generative adversarial networks.

Generally, it is difficult to obtain high resolution images with generative adversarial neural networks (GANs). In order to overcome this disadvantage, the method according to the invention advantageously can comprise a prior step of reducing the size of the majority and minority real medical images. For example, the medical images can be reduced to a size of 128×128 pixels (if a two-dimensional image is involved) and can be centred on the anatomy of interest.

In particular embodiments, the real medical images from which the majority segmentation masks and the minority segmentation masks are generated are medical images obtained by tomodensitometry, by positron emission tomography, by magnetic resonance imaging or by ultrasound.

In particular embodiments, the anatomy of interest is an organ such as the liver, a lung or a kidney, or another anatomical structure such as a bone or a blood vessel.

In particular embodiments, the anomaly is a tumour or an ablation zone.

According to a second aspect, a method for training a machine learning algorithm aiming to detect or to characterize an anomaly in the anatomy of interest of a patient on a real medical image is proposed. The method comprises generating, using a method according to any one of the preceding embodiments, synthetic medical images representing the anatomy of interest and an anomaly within said anatomy of interest. The method subsequently comprises training the machine learning algorithm using a set of training images comprising the synthetic medical images thus generated.

In particular embodiments, the method can further comprise one or more of the following feature(s), taken individually or according to all the technically possible combinations.

In particular embodiments, the set of training images comprises synthetic medical images and real medical images, and the number of images with an anomaly is at least equal to 10% of the number of images without an anomaly.

In particular embodiments, the machine learning algorithm is an anomaly classification algorithm.

In particular embodiments, the machine learning algorithm is an anomaly segmentation algorithm.

In particular embodiments, the machine learning algorithm is implemented by a deep neural network.

According to a third aspect, a method for detecting or for characterizing an anomaly in the anatomy of interest of a patient on a real medical image is proposed. The method comprises:
training, using a method according to any one of the preceding embodiments, a machine learning algorithm aiming to detect or to characterize an anomaly in the anatomy of interest of a patient on a real medical image;
receiving a real medical image of the anatomy of interest of a patient;
analyzing said real medical image with the trained machine learning algorithm;
obtaining, as output from the trained machine learning algorithm, information allowing an anomaly in the anatomy of interest visible on the real medical image to be detected or to be characterized.

According to a fourth aspect, a device comprising one or more processor(s) and at least one storage medium that can be read by the one or more processor(s) is proposed. The storage medium being intended to store majority segmentation masks and minority segmentation masks. Each majority segmentation mask comprises a segmentation of an anatomy of interest visible on a majority real medical image of the anatomy of interest of a patient without an anomaly. Each minority segmentation mask comprises a segmentation of an anomaly visible on a minority real medical image of the anatomy of interest of a patient with an anomaly within said anatomy of interest. The storage medium comprises a set of program code instructions that, when the program is executed by the one or more processor(s), configure the one or more processor(s) in order to generate a set of artificial segmentation masks from the majority segmentation masks and from the minority segmentation masks stored on the storage medium. Each artificial segmentation mask is generated by combining the segmentation of the anatomy of interest of a majority segmentation mask with the segmentation of the anomaly of a minority segmentation mask.

In particular embodiments, the device can further comprise one or more of the following feature(s), taken individually or according to all the technically possible combinations.

In particular embodiments, in order to generate an artificial segmentation mask, the one or more processor(s) is/are also configured to transform the segmentation of the anomaly of the minority segmentation mask.

In particular embodiments, the transformation of the segmentation of the anomaly corresponds to a rotation, a magnification, a reduction, a deformation or a movement of the segmentation of the anomaly.

In particular embodiments, in order to generate an artificial segmentation mask, the one or more processor(s) is/are also configured to check that the segmentation of the anomaly relative to the segmentation of the anatomy of interest meets a particular criterion.

In particular embodiments, a segmentation mask comprises a set of voxels. Each voxel corresponds to a zone of the real medical image with which the segmentation mask is associated. Each voxel is associated with a numerical value encoding what is shown by said zone on the real medical image. In order to generate an artificial segmentation mask, the one or more processor(s) is/are configured to:
select a majority segmentation mask and a minority segmentation mask;
identify, on the selected minority segmentation mask, a set of voxels, the numerical value of which encodes the anomaly;
replace, on the selected majority segmentation mask, the numerical value of the voxels identified by the numerical value encoding the anomaly.

In particular embodiments, the storage medium also stores a neural network previously trained to generate a synthetic medical image from a segmentation mask and, when the program is executed, the one or more processor(s) is/are configured to generate synthetic medical images with the neural network from artificial segmentation masks.

In particular embodiments, the neural network for generating a synthetic medical image is a generator neural network adapted to be trained using a discriminator neural network, and the pair formed by the generator neural network and the discriminator neural network form a pair of generative adversarial networks.

### DESCRIPTION OF THE FIGURES

The invention will be better understood from reading the following description, which is provided by way of a non-limiting example, and with reference to FIGS. 1 to 14, which show:

FIG. 1 a schematic representation of the main steps of a method according to the invention for generating synthetic medical images representing an anatomical anomaly;

FIG. 2 a schematic representation of the main steps of a method according to the invention for training a machine learning algorithm aiming to detect or to characterize an anomaly in the anatomy of interest of a patient on a real medical image;

FIG. 3 a schematic representation of the main steps of a method according to the invention for detecting or for characterizing an anomaly in the anatomy of interest of a patient on a real medical image;

FIG. 4 a schematic representation of a step of generating majority segmentation masks from majority real medical images;

FIG. 5 a schematic representation of a step of generating minority segmentation masks from minority real medical images;

FIG. 6 a schematic representation of a step of generating artificial segmentation masks from majority segmentation masks and from minority segmentation masks;

FIG. 7 a schematic representation of a step of generating artificial segmentation masks comprising a transformation of the segmentation of the anomaly;

FIG. 8 an illustration of the generation of an artificial segmentation mask from a majority segmentation mask and from a minority segmentation mask;

FIG. 9 a schematic representation of a step of training a neural network to generate a synthetic medical image from a segmentation mask;

FIG. 10 an illustration of a real medical image, of its associated segmentation mask, and of a synthetic medical image generated by the neural network from the segmentation mask, for a majority case (without an anomaly) and a minority case (with an anomaly);

FIG. 11 a schematic representation of a step of generating a synthetic medical image from an artificial segmentation mask using the previously trained neural network;

FIG. 12 an illustration, for three different majority real medical images, of the majority real medical image, of its associated majority segmentation mask, of an artificial segmentation mask generated from the majority segmentation mask and from a minority segmentation mask, and of a synthetic medical image generated by the neural network from the artificial segmentation mask;

FIG. 13 a schematic representation of a step of training, from a synthetic medical image, the machine learning algorithm;

FIG. 14 a schematic representation of the detection or of the characterization of an anomaly in an anatomy of interest shown on a real medical image by the machine learning algorithm.

Throughout these figures, identical reference signs from one figure to another denote identical or similar elements. For the sake of clarity, the elements that are shown are not necessarily to the same scale, unless otherwise stated.

### DETAILED DESCRIPTION OF AT LEAST ONE EMBODIMENT OF THE INVENTION

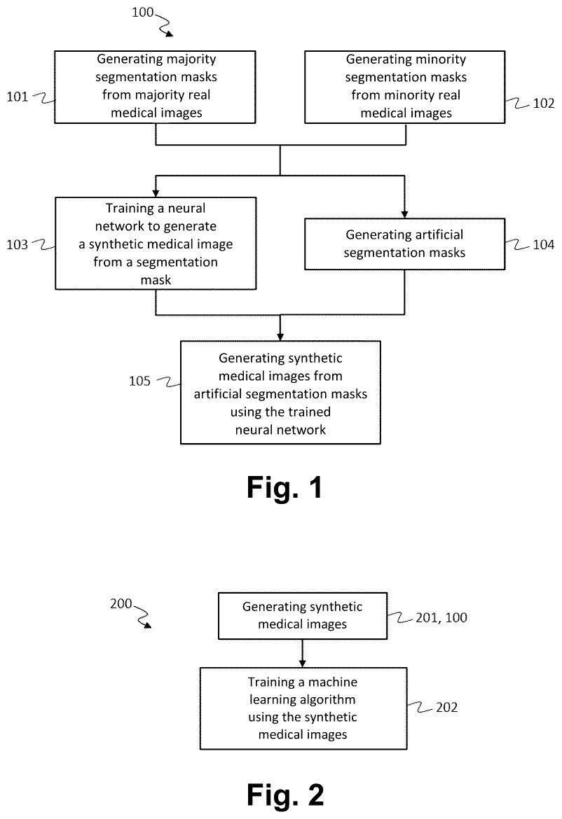

FIG. 1 schematically shows the main steps of a method 100 according to the invention for generating synthetic medical images representing an anomaly in an anatomy of interest.

Throughout the remainder of the description, the anatomy of interest will be considered, by way of a non-limiting example, to be the liver and the anomaly will be considered to be a tumour. However, it should be noted that the method could be applied to other anatomies of interest, such as, for example, a lung, a kidney, a bone, a blood vessel, etc. Furthermore, the method could be applied to other types of anomalies, such as, for example, a tumour, a cyst, an ablation zone, an aneurysm, etc.

The method 100 comprises a step 101 of generating majority segmentation masks. Each majority segmentation mask is associated with a majority real medical image representing the anatomy of interest of a patient in a case whereby said anatomy of interest does not exhibit an anomaly.

Throughout the remainder of the description, a real medical image is considered to be a two-dimensional image acquired by a tomodensitometry imaging device. However, in variants, nothing prevents the use of three-dimensional real medical images or of real medical images acquired using other imaging modes, such as, for example, by magnetic resonance, by positron emission tomography, by ultrasound, or by x-rays.

The method 100 also comprises a step 102 of generating minority segmentation masks. Each minority segmentation mask is associated with a minority real medical image representing the anatomy of interest of a patient in a case whereby said anatomy of interest exhibits an anomaly.

It should be noted that the order of steps 101 and 102 is unimportant. Furthermore, the method 100 can optionally comprise a prior step of reducing the size of the real medical images. For example, the medical images can be reduced to a size of 128×128 pixels (if it is a two-dimensional image) and can be centred on the anatomy of interest, before being used to generate the segmentation masks.

The method 100 comprises a step 103 of training a neural network 31 for generating a synthetic medical image from a segmentation mask. In order to train the neural network, the majority segmentation masks generated in step 101 and/or the minority segmentation masks generated in step 102 can be used, for example. However, other segmentation masks also can be used.

Throughout the remainder of the description, the neural network used to generate a synthetic medical image from a segmentation mask is considered to be a generator of a pair of generative adversarial neural networks (GAN). However, it should be noted that it would also be possible to use other types of neural networks, such as, for example, an autoencoder type neural network (VAE). The selection of a particular type of neural network for generating a synthetic medical image from a segmentation mask is only one variant of the invention.

The method 100 comprises a step 104 of generating artificial segmentation masks from majority segmentation masks and from minority segmentation masks. The generation 104 of an artificial segmentation mask is based on combining a segmentation of the anatomy of interest of a majority segmentation mask with a segmentation of the anomaly 25 of a minority segmentation mask.

It should be noted that steps 103 and 104 are independent of each other and that the order in which they are executed is not important.

Finally, the method 100 comprises a step 105 of generating synthetic medical images from artificial segmentation masks using the previously trained neural network.

Steps 101 to 105 will be described hereafter with reference to FIGS. 4 to 12.

FIG. 2 schematically shows the main steps of a method 200 according to the invention for training a machine learning algorithm aiming to detect or to characterize an anomaly in the anatomy of interest of a patient on a real medical image.

The method 200 particularly comprises a step 201 of generating synthetic medical images representing the anatomy of interest and an anomaly within said anatomy of interest. The generation 201 of synthetic medical images is implemented using the method 100 described above with reference to FIG. 1.

The method 200 subsequently comprises a step 202 of training the machine learning algorithm using a set of training images comprising the synthetic medical images generated in step 201.

The machine learning algorithm corresponds, for example, to a deep neural network. However, nothing prevents the use of other types of machine learning algorithms, such as, for example, a “random forest” algorithm.

The machine learning algorithm corresponds, for example, to a classification algorithm that allows the nature of the anomaly (type of tumour) to be identified. According to another example, the machine learning algorithm corresponds to a segmentation algorithm that allows the outline of the anomaly on a medical image to be defined.

The set of training images can comprise both real medical images and synthetic medical images. The number of medical images exhibiting an anomaly and the variety of the anomalies are considerably increased by virtue of the wide variety of artificial segmentation masks used to generate the synthetic medical images. Preferably, the number of images with an anomaly is at least equal to 10% of the number of images without an anomaly. Ideally, the set of training images comprises a number of images with an anomaly that is substantially equal to the number of images without an anomaly. This thus allows the phenomenon of overlearning to be avoided on a particular class of anomaly.

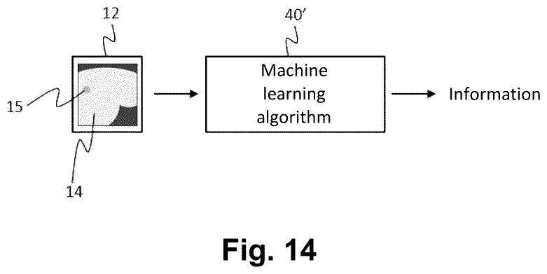

The step 202 of training the machine learning algorithm will be described hereafter with reference to FIG. 13.

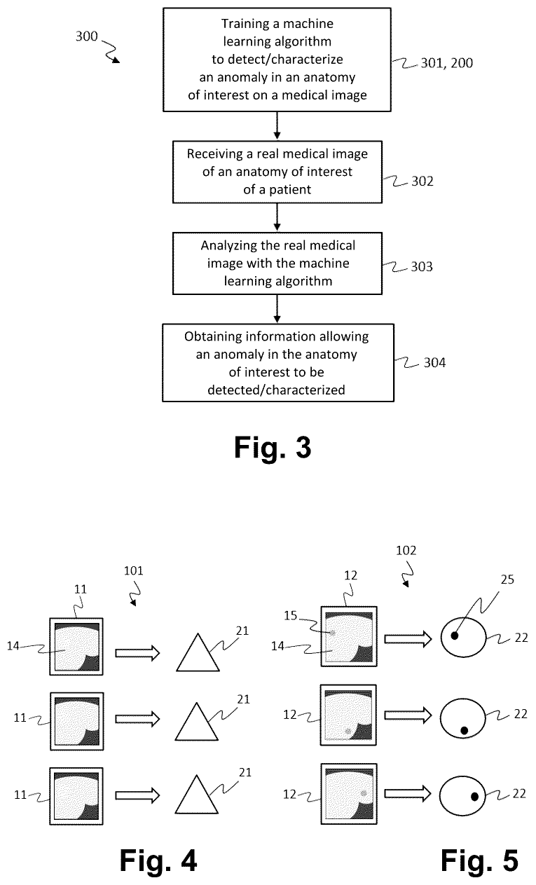

FIG. 3 schematically shows the main steps of a method 300 according to the invention for detecting or for characterizing an anomaly in the anatomy of interest of a patient on a real medical image.

The method 300 particularly comprises a step 301 of training a machine learning algorithm aiming to detect or to characterize an anomaly in the anatomy of interest of a patient on a real medical image. This training step 301 is implemented using the method 200 described above with reference to FIG. 2.

The method 300 subsequently successively comprises a step 302 of receiving a real medical image of the anatomy of interest of a patient, a step 303 of analyzing said real medical image with the trained machine learning algorithm, and a step 304 of obtaining, as output from the trained machine learning algorithm, information allowing the detection or the characterization of an anomaly in the anatomy of interest visible on the real medical image.

FIG. 4 schematically shows the step 101 of generating a majority segmentation mask 21 from a majority real medical image 11. The anatomy of interest 14 is visible on the majority real medical image 11. On a real medical image, called “majority” image, there is no anomaly within the anatomy of interest. The majority segmentation mask 21 comprises a segmentation of the anatomy of interest.

FIG. 5 schematically shows the step 102 of generating a minority segmentation mask 22 from a minority real medical image 12. The anatomy of interest 14 is visible on the minority real medical image 12. On a real medical image, called “minority” image, an anomaly 15 is present within the anatomy of interest 14. The minority segmentation mask 22 comprises a segmentation of the anatomy of interest and a segmentation of the anomaly 25.

In medical imaging, segmentation is an essential step that involves extracting one or more particular anatomical region(s) from an image. A segmentation mask is an image for which each voxel (or pixel in the case of a two-dimensional image) provides particular information relating to an element shown at the position of the voxel on the real medical image. A voxel thus can assume a particular numerical value associated with the element shown at the position of the voxel on the real medical image. Different specific numerical values are respectively defined, for example, for a healthy part of the anatomy of interest, for an anomaly within the anatomy of interest, for other anatomical structures (for example, bones, blood vessels), for the background of the image, etc.

Segmentation can be implemented manually. In this case, the practitioner defines the outlines of the various anatomical regions on a medical image themselves using a graphics interface (mouse, stylus, touchscreen, etc.) of an electronic device (computer, tablet, etc.) on which the image is displayed. Segmentation also can be implemented automatically, using an artificial intelligence segmentation algorithm.

For example, the bones visible on the medical images are segmented using the voxels intensity thresholding method with an intensity window with a width of 1800 HU and a centre of 400 HU (HU is the acronym for “Houndsfield Unit”, it is a quantitative scale describing the radio density, i.e. a measuring unit representing the opacity of a material to a radio wave).

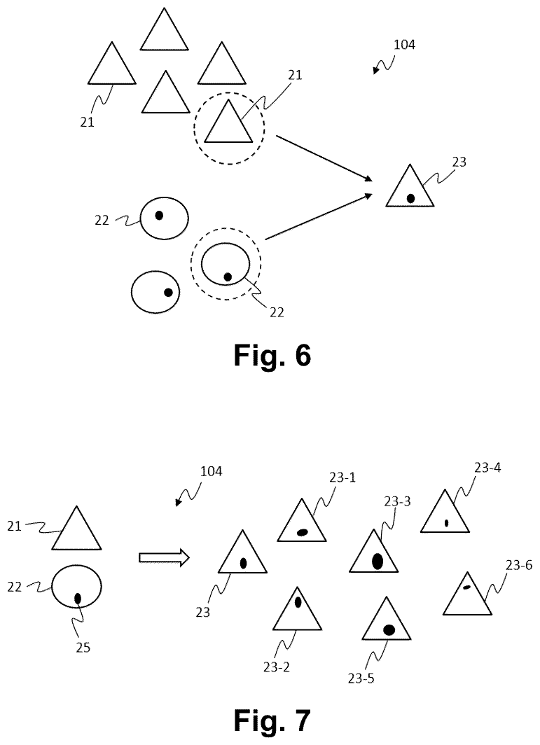

FIG. 6 schematically shows the step 104 of generating artificial segmentation masks 23 from majority segmentation masks 21 and from minority segmentation masks 22.

In the example illustrated in FIG. 6, the step 104 of generating an artificial segmentation mask 23 comprises:
selecting a majority segmentation mask 21;
selecting a minority segmentation mask 22;
identifying, on the selected minority segmentation mask 22, a set of voxels, the numerical value of which encodes the anomaly;
replacing, on the selected majority segmentation mask 21, the numerical value of the voxels identified by the numerical value encoding the anomaly.

The majority segmentation mask 21 and the minority segmentation mask 22 can be selected randomly. The number of combinations of artificial segmentation masks thus can amount to the product of the number of majority segmentation masks 21 by the number of minority segmentation masks 22.

A plurality of minority segmentation masks 21 with various semantic values (i.e. with various types of anomaly: tumour, cyst, ablation region, artefact, etc.) can be combined with majority segmentation masks 22 and thus increase the number of potential combinations. Manipulation of the proportions of the various minority masks 21 used in the combinations of artificial segmentation masks 23 allows the characteristics of the synthetic images produced to be controlled. Likewise, a plurality of majority masks 22 with various semantic values (liver, lung, pancreas, gall bladder, etc.) can be combined with the minority segmentation masks 21. These various majority masks 22 make it possible to control in which organ or structure the minority segmentation masks 21 can appear by virtue of Boolean logic rules (for example rules such as “AND”, “OR”, “NOT”) between the various majority masks 22 and minority masks 21 for each pixel of the artificial segmentation masks 23. This feature for example allows in which organ the random distribution of a minority segmentation mask 21 of a tumour can appear (“AND”) and in which structure or organ this minority segmentation mask 21 cannot appear (“NOT”) to be controlled.

The combinations of majority segmentation mask 21 and of minority segmentation mask 22 of greatest interest also can be selected as a function of a distance estimated between a segmentation of the anomaly 25 on the minority segmentation mask 22 and a segmentation of interest of the majority segmentation mask 21, such as, for example, the segmentation of the gall bladder, the vessels, the hilum, or even the liver capsule.

As illustrated in FIG. 7, the step 104 of generating artificial segmentation masks 23 can comprise a transformation of the segmentation of the anomaly 25 of the selected minority segmentation mask 22. Such arrangements allow a larger number of different artificial segmentation masks to be generated. The transformation of the segmentation of the anomaly 25 corresponds, for example, to a rotation (as illustrated for the artificial segmentation mask 23-1), a movement (as illustrated for the artificial segmentation mask 23-2), a magnification (as illustrated for the artificial segmentation mask 23-3), a reduction (as illustrated for the artificial segmentation mask 23-4), a deformation (as illustrated for the artificial segmentation mask 23-5) or a combination of these various possible transformations (as illustrated for the artificial segmentation mask 23-6 for which the segmentation of the anomaly 25 was equally moved, reduced and turned).

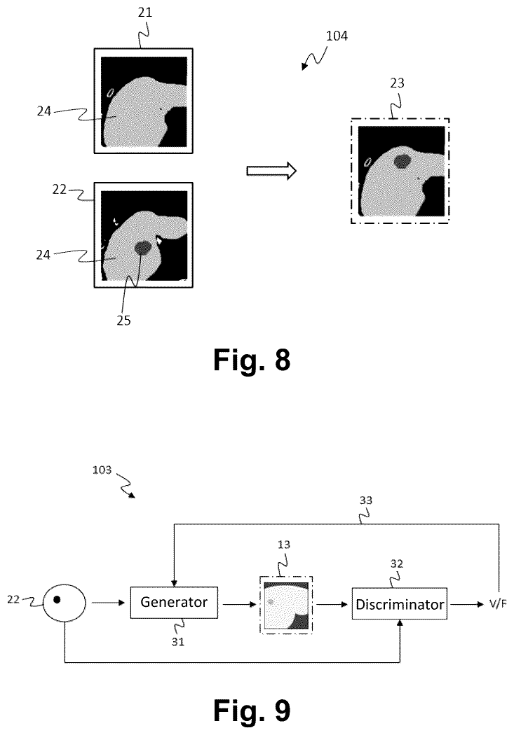

FIG. 8 illustrates the generation 104 of an artificial segmentation mask 23 from a majority segmentation mask 21 and from a minority segmentation mask 22. The segmentation of the anatomy of interest 24-1 can be seen on the majority segmentation mask 21. Both the segmentation of the anatomy of interest 24-2 and the segmentation of the anomaly 25-2 can be seen on the minority segmentation mask 22. The segmentation of the anatomy of interest 24-1 of the majority segmentation mask 21 combined with the segmentation of the anomaly 25-2 of the minority segmentation mask 22 (in the considered example, the segmentation of the anomaly 25-2 has also been moved) can be seen on the generated artificial segmentation mask 23.

In particular embodiments, generating 104 an artificial segmentation mask 23 can further comprise a step of checking that the segmentation of the anomaly 25-2 relative to the segmentation of the anatomy of interest 24-1 meets a particular criterion.

This additional checking step ensures that certain particular constraints are met. This can particularly allow certain unrealistic combinations to be filtered. These constraints relate to, for example, the location of the anomaly within the organ of interest or relative to other organs or other anatomical structures. Thus, by way of an example, the criterion can correspond to the fact that a distance between an edge of the segmentation of the anatomy of interest and an edge of the segmentation of the anomaly must be at least equal to a threshold value. However, this depends on the targeted application: if seeking to synthetize an image of a tumour in an organ, then provision will be made to ensure that the anomaly is located in the organ; however, if seeking to synthetize an image with tumours in various organs, superposition of the segmentations of the tumours with the segmentations of various organs will be authorized.

This generation 104 of the artificial segmentation masks 23 can be implemented by an electronic device, such as a computer, for example. The device comprises, for example, one or more processor(s) and at least one storage medium that can be read by the one or more processor(s). The storage medium is intended to store the majority segmentation masks 21 and the minority segmentation masks 22. The storage medium further comprises a set of program code instructions, which, when the program is executed by the one or more processor(s), configure the one or more processor(s) in order to generate, as described above with reference to FIGS. 6 to 8, a set of artificial segmentation masks 23 from the majority segmentation masks 21 and from the minority segmentation masks 22 stored on the storage medium.

FIG. 9 schematically shows the step 103 of training a neural network 31 to generate a synthetic medical image 13 from a segmentation mask 22.

In the considered example, the neural network 31 used to generate a synthetic medical image 13 is a generator neural network 31, and the training of the generator neural network 31 is implemented using a discriminator neural network 32. The generator neural network 31 and the discriminator neural network 32 form a pair of generative adversarial networks (GAN). The GAN type of neural networks actually allows highly realistic images to be generated. In a GAN, the generator neural network and the discriminator neural network are set to compete in a zero-sum game scenario. The generator generates an image, and their opponent, the discriminator, attempts to detect whether the generated image is real or if it is a synthetic image generated by the generator.

Firstly, the generator 31 is trained to generate synthetic images 13 from the segmentation masks. The segmentation masks at the input of the generator 31 equally can be minority segmentation masks 22 (as in the example illustrated in FIG. 9) or majority segmentation masks 21.

Secondly, the synthetic images 13 generated by the generator are analyzed by the discriminator 32, which has been previously trained to recognize, by taking an image 13 and an associated segmentation mask 22 as input, whether the pair formed by the image 13 and the segmentation mask 22 is real. Therefore, at the output of the discriminator 32, a “True” or “False” decision is taken whereby the pair formed by the image 13 and the segmentation mask 22 is considered to be real or false. Using a back-propagation loop 33, as a function of the veracity of the decision taken by the discriminator 32, the parameters of the generator 31 are modified until the generated synthetic images 13 relating to the segmentation mask 22 are considered to be real by the discriminator 32.

The generator 31 is an image-to-image translation neural network. It is, for example, a convolutive type “pix2pix” neural network, as described in document “Image-to-Image translation with conditional adversarial networks” by Isola, P et al. In the considered example, the neural network comprises a first part of the encoder type made up of “Batch Normalisation Leaky ReLU” convolution layers with 4×4 convolution filters of sizes 64, 128, 256, 512, 512, 512, 512 and a second part of the decoder type made up of “Batch Normalisation Dropout ReLU” convolution layers with 4×4 convolution filters of sizes 512, 512, 512, then of “Batch Normalisation ReLU” convolution layers with 4×4 convolution filters of sizes 256, 128, 64. The image size reduction in the encoder part is produced by pixel “strides” of two and the increase in the size of the image in the decoder part is produced by virtue of a 2D magnification layer (“Upsampling2D”, “Nearest Neighbours” method) with a size of 2×2. The output is produced by virtue of a hyperbolic tangent type activation layer (Tanh).

The discriminator 32 is a neural network. For example, it is a “PatchGAN” type convolutive neural network as described in the document “Image-to-Image translation with conditional adversarial networks” by Isola, P et al, modified to accept two images as input that are concatenated into a single image. The remainder of the neural network is made up of a “Leaky ReLU” convolution layer with 4×4 convolution filters of size 64, then of four “Batch Normalisation Leaky ReLU” convolution layers with 4×4 convolution filters of sizes 128, 256, 512, 512. The output is produced using a sigmoid activation layer.

The image-to-image translation generator neural network 31 (“pix2pix”) is combined with the PatchGAN type discriminator 32 in such a way that the output predictions of the generator 31 (the synthetic medical images 13) form the second input of the discriminator 32. The first input of the discriminator corresponds to the segmentation mask 22 that is provided as input for the generator 31. The output is a 70×70 probability matrix. The weights of the neurons of the discriminator 32 cannot be modified when training the generator 31. The weights of the generator 31 can be updated during training. The cost computation function is made up of the cross-entropy and the norm 1 in a ratio of 1:100.

The discriminator 32 and the generator 31 are alternately trained, in turn, on a training set comprising majority pairs (each majority pair comprises a majority medical image and the associated majority segmentation mask) and minority pairs (each minority pair comprises a minority medical image and the associated minority segmentation mask). The output of the discriminator 32 is optimized using the Adam-type stochastic gradient algorithm (Adaptive Moment Estimation, beta_1:0.9, beta_02:0. 999, epsilon: 1e-08) against a 70×70 matrix of 1 values when its input is a “true” pair (i.e. a pair comprising a segmentation mask and its associated real medical image), and of 0 values when its input is a “false” pair (i.e. a pair comprising a segmentation mask and a synthetic medical image produced by the generator 31 from the segmentation mask). The output of the generator 31 is optimized using the Adam-type stochastic gradient algorithm against a 70×70 matrix of 1 values so that the weights of the neurons of the generator 31 are updated, but not those of the discriminator 32, when the discriminator 32 detects that the input pair is not close enough to the “true” pairs already encountered. Alternately updating the weights of the discriminator 32 allows it to be ahead of the generator 31 and compels it to update itself.

An independent neural network can be used to control the training of the adversarial networks 31, 32. For example, the pre-trained “InceptionV3” model can be used, after having removed the last classification layer, to compare the synthetic images 13 produced by the generator 31 and the real images 12 (“InceptionV3” is a convolutive neural network for assisting image analysis). The activation values produced as output from the model by the two images are used to compute an FID score (acronym for “Frechet Inception Distance”). The lower this score, the more similar the images. The weights of the generator 31 are saved as soon as the new FID score is lower than the previous recorded score. The training is stopped when the FID score becomes too large compared to the minimum obtained during the training.

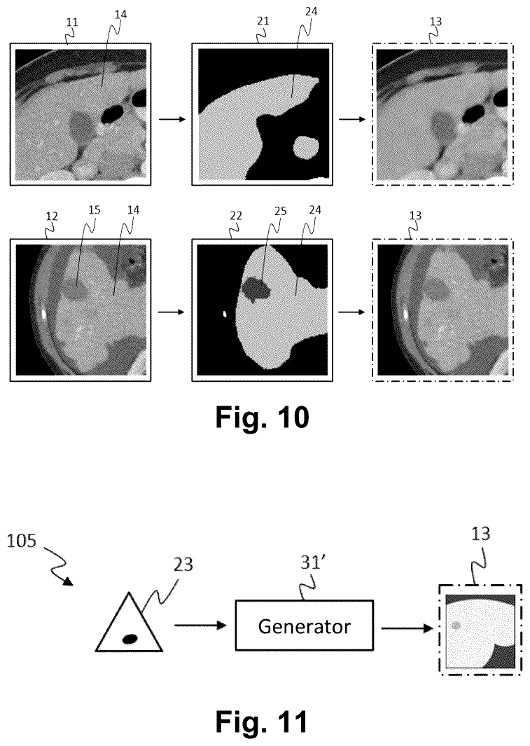

FIG. 10 is an illustration of a real medical image 11, 12, its associated segmentation mask 21, 22, and a synthetic medical image 13 generated by the neural network 31 from the segmentation mask 21, 22, for a majority case (without an anomaly) and a minority case (with an anomaly).

Once the training of the neural network 31 is complete, and as illustrated in FIG. 11, the trained neural network 31′ can be used to generate synthetic medical images 13 from the artificial segmentation masks 23.

The device that implements the step 104 of generating the artificial segmentation masks 23 can also implement the step 105 of generating the synthetic medical images 13. In this case, the storage medium of the device stores the previously trained generator neural network 31′ and, when the program is executed, the one or more processor(s) of the device are configured to generate synthetic medical images 13 with the neural network 31′ from artificial segmentation masks 23.

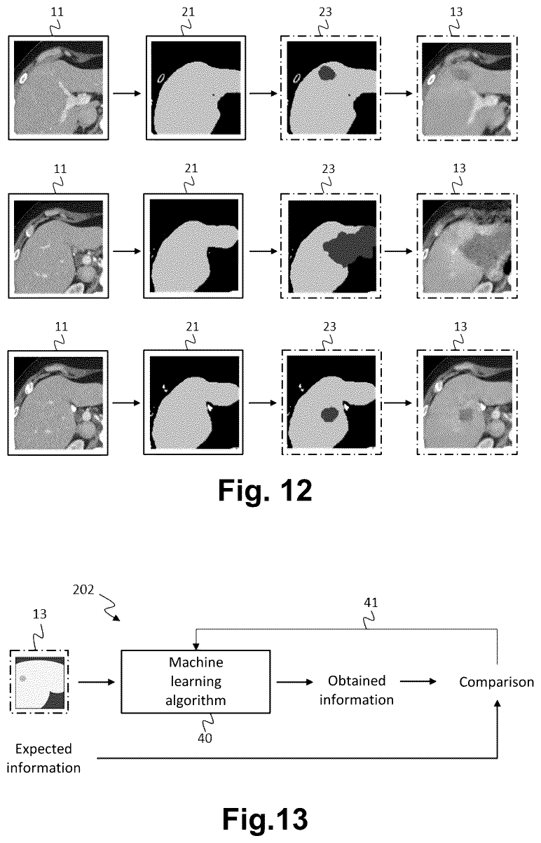

FIG. 12 illustrates, by way of an example, for three different majority real medical images 11: the majority real medical image 11, its associated majority segmentation mask 21, an artificial segmentation mask 23 generated from the majority segmentation mask, and a synthetic medical image 13 generated by the neural network 31′ from the artificial segmentation mask 23.

Due to the wide variety of artificial segmentation masks 23 generated in step 104, a wide variety of synthetic medical images 13 exhibiting anomalies can be generated. This diversity of the synthetic medical images 13 allows a set of training images to be generated that is particularly effective for a machine learning algorithm aiming to detect or to characterize an anomaly in the anatomy of interest of a patient on a real medical image. The set of training images can comprise both real medical images and synthetic medical images. Preferably, the set of training images comprises a number of images with an anomaly that is substantially equal to the number of images without an anomaly. This thus avoids the phenomenon of overlearning on a particular class of anomaly.

FIG. 13 schematically shows the step 202 of training the machine learning algorithm 40 from a synthetic medical image 13. The machine learning algorithm 40 aims to detect or to characterize an anomaly in the anatomy of interest of a patient on a medical image. In the considered example, the machine learning algorithm 40 is a deep neural network.

As illustrated in FIG. 13, during the training phase the machine learning algorithm 40 takes a medical image as input (it is a synthetic medical image 13 on the example illustrated in FIG. 13). The information that must be obtained as output from the machine learning algorithm 40 is a priori known (“expected information”). The obtained information and the expected information are compared and, depending on the result of the comparison, the parameters of the neural network are updated by a back-propagation loop 41. The training continues until the machine learning algorithm 40 is capable of providing the expected information with a satisfactory success rate.

FIG. 14 schematically shows the detection or the characterization of an anomaly 15 in an anatomy of interest 14 shown on a real medical image 12 by the machine learning algorithm 40′ thus trained. The information supplied as output from the machine learning algorithm 40′ corresponds, for example, to an indication that an anomaly has been detected, to a classification of the anomaly (nature of the tumour, for example) and/or to a segmentation of the anomaly on the medical image 12.

In one alternative operating mode, the neural network 31 is trained at the same time as the machine learning algorithm 40 is trained. In this operating mode, the neural network 31 never ceases being trained and the synthetic images are produced by the neural network 31 and used by the machine learning algorithm 40 at regular intervals without an intermediate storage step. Each batch of synthetic images is produced by the neural network 31 in various training steps, and therefore a given artificial segmentation mask 23 will produce a different synthetic image. Operating in this way allows an infinite variability in the images produced by the neural network 31 for the machine learning algorithm 40. This feature is important because it limits over-fitting by the machine learning algorithm 40 to the training data, as the algorithm will never use the same synthetic image twice during training.

1. A method for generating synthetic medical images representing an anatomy of interest and an anomaly within said anatomy of interest, said method comprising:
generating majority segmentation masks, each majority segmentation mask being associated with a majority real medical image representing the anatomy of interest of a patient without an anomaly;
generating minority segmentation masks, each minority segmentation mask being associated with a minority real medical image representing the anatomy of interest of a patient with an anomaly;
training a neural network to generate a synthetic medical image from a segmentation mask;
generating artificial segmentation masks from majority segmentation masks and from minority segmentation masks, the generation of an artificial segmentation mask comprising combining a segmentation of the anatomy of interest of a majority segmentation mask with a segmentation of the anomaly of a minority segmentation mask; and
generating synthetic medical images from the artificial segmentation masks using the previously trained neural network.

2. The method according to claim 1, wherein the generation of an artificial segmentation mask further comprises transforming the segmentation of the anomaly of the minority segmentation mask.

3. The method according to claim 2, wherein the transformation of the segmentation of the anomaly corresponds to a rotation, a magnification, a reduction, a deformation and/or a movement of the segmentation of the anomaly.

4. The method according to claim 1, wherein the generation of an artificial segmentation mask further comprises checking that the segmentation of the anomaly relative to the segmentation of the anatomy of interest meets a particular criterion.

5. The method according to claim 1, wherein a segmentation mask comprises a set of voxels, with each voxel corresponding to a zone of the real medical image with which the segmentation mask is associated, with each voxel being associated with a numerical value encoding what is shown by said zone on the real medical image, and the step of generating an artificial segmentation mask comprises:
selecting a majority segmentation mask and a minority segmentation mask;
identifying, on the selected minority segmentation mask, a set of voxels, the numerical value of which encodes the anomaly; and
replacing, on the selected majority segmentation mask, the numerical value of the voxels identified by the numerical value encoding the anomaly.

6. The method according to claim 1, wherein the neural network used to generate a synthetic medical image is a generator neural network, and the training of the generator neural network is implemented using a discriminator neural network, with the generator neural network and the discriminator neural network forming a pair of generative adversarial networks.

7. The method according to claim 1, wherein the real medical images from which the majority segmentation masks and the minority segmentation masks are generated are medical images obtained by tomodensitometry, by positron emission tomography, by magnetic resonance imaging or by ultrasound.

8. The method according to claim 1, wherein the anatomy of interest is a liver, a lung, a kidney, a bone or a blood vessel.

9. The method according to claim 1, wherein the anomaly is a tumour or an ablation zone.

10. A method for training a machine learning algorithm aiming to detect or to characterize an anomaly in the anatomy of interest of a patient on a real medical image, said method comprising:
generating, using a method according to claim 1, synthetic medical images representing the anatomy of interest and an anomaly within said anatomy of interest; and
training the machine learning algorithm using a set of training images comprising the synthetic medical images thus generated.

11. The method according to claim 10, wherein the set of training images comprises synthetic medical images and real medical images, and the number of images with an anomaly is at least equal to 10% of the number of images without an anomaly.

12. The method according to claim 10, wherein the machine learning algorithm is an anomaly classification algorithm.

13. The method according to claim 10, wherein the machine learning algorithm is an anomaly segmentation algorithm.

14. The method according to claim 10, wherein the machine learning algorithm is implemented by a deep neural network.

15. A device comprising one or more processor(s) and at least one storage medium that can be read by the one or more processor(s), the storage medium being intended to store majority segmentation masks and minority segmentation masks, each majority segmentation mask comprising a segmentation of an anatomy of interest visible on a majority real medical image of the anatomy of interest of a patient without an anomaly, each minority segmentation mask comprising a segmentation of an anomaly visible on a minority real medical image of the anatomy of interest of a patient with an anomaly within said anatomy of interest, wherein the storage medium comprises a set of program code instructions which, when the program is executed by the one or more processor(s), configure the one or more processor(s) in order to generate a set of artificial segmentation masks from the majority segmentation masks and from the minority segmentation masks stored on the storage medium, with each artificial segmentation mask being generated by combining the segmentation of the anatomy of interest of a majority segmentation mask with the segmentation of the anomaly of a minority segmentation mask.

16. The device according to claim 15, wherein, in order to generate an artificial segmentation mask, the one or more processor(s) is/are also configured to transform the segmentation of the anomaly of the minority segmentation mask.

17. The device according to claim 16, wherein the transformation of the segmentation of the anomaly corresponds to a rotation, a magnification, a reduction, a deformation or a movement of the segmentation of the anomaly.

18. The device according to claim 15, wherein, in order to generate an artificial segmentation mask, the one or more processor(s) is/are also configured to check that the segmentation of the anomaly relative to the segmentation of the anatomy of interest meets a particular criterion.

19. The device according to claim 15, wherein a segmentation mask comprises a set of voxels, with each voxel corresponding to a zone of the real medical image with which the segmentation mask is associated, with each voxel being associated with a numerical value encoding what is shown by said zone on the real medical image and, in order to generate an artificial segmentation mask, the one or more processor(s) is/are configured to:
select a majority segmentation mask and a minority segmentation mask;
identify, on the selected minority segmentation mask, a set of voxels, the numerical value of which encodes the anomaly; and
replace, on the selected majority segmentation mask, the numerical value of the voxels identified by the numerical value encoding the anomaly.

20. The device according to claim 15, wherein the storage medium also stores a neural network previously trained to generate a synthetic medical image from a segmentation mask and, when the program is executed, the one or more processor(s) is/are configured to generate synthetic medical images with the neural network from artificial segmentation masks.

21. The device according to claim 20, wherein the neural network for generating a synthetic medical image is a generator neural network adapted to be trained using a discriminator neural network, the generator neural network and the discriminator neural network forming a pair of generative adversarial networks.
